(19) United States
(12) Patent Application Publication
(10) Pub. No.: US 2024/0245649 A1
(43) Pub. Date: Jul. 25, 2024

(54) Method and Composition for Mimicking Caloric Restriction by Administration of Ergothioneine

(71) Applicant: NANJING NUTRABUILDING BIO-TECH CO., LTD., Nanjing (CN)

(72) Inventors: Mingru WANG, Nanjing (CN); Ronghua YI, Nanjing (CN); Shawn WELLS, Nanjing (CN); Kylin LIAO, Nanjing (CN)

(21) Appl. No.: 18/626,324

(22) Filed: Apr. 4, 2024

Related U.S. Application Data
(63) Continuation of application No. PCT/CN2022/122527, filed on Sep. 29, 2022.

(30) Foreign Application Priority Data
Oct. 8, 2021 (WO) PCT/CN2021/122680

(51) Int. Cl. A61K 31/4172; A61P 3/06; A61P 3/10

(52) U.S. Cl. CPC A61K 31/4172; A61P 3/06; A61P 3/10

(57) ABSTRACT

Among others, the present invention provides methods and compositions for mimicking one or more biological benefits of caloric restriction in a mammal, including administering to the mammal an effective amount of Ergothioneine, an analog or derivative thereof, or a pharmaceutically acceptable salt, ester, polymer, acid, analog or derivative thereof.

### CROSS REFERENCE TO RELATED APPLICATION

This application is a continuation application of International Patent Application No. PCT/CN2022/122527, filed on Sep. 29, 2022, which claims the priority of the international application No. PCT/CN2021/122680, filed on Oct. 8, 2021, the contents of all of which are incorporated herein by reference in their entirety.

### FIELD OF THE INVENTION

This invention generally relates to the field of methods for mimicking caloric restriction in a mammal, and more specifically, relates to methods and compositions for mimicking the biological benefits of caloric restriction in a mammal, involving administering to the mammal an effective amount of Ergothioneine, an analog or derivative thereof, or a pharmaceutically acceptable salt, ester, polymer, acid, analog or derivative thereof.

### BACKGROUND OF THE INVENTION

Caloric restriction (CR), also known as dietary restriction, is the reduction of food intake without malnutrition. Studies in numerous species have demonstrated that reduction of calories 30-50% below ad libitum levels of a nutritious diet can increase lifespan, reduce the incidence and delay the onset of age-related diseases, improve stress resistance, and decelerate functional decline (Aging Cell. 2006 April; 5(2):97-108). There are various calorie-restricted diets. The general principle is to eat low-calorie foods, such as vegetables and fruits, and to avoid eating some higher-calorie foods, such as starches and fatty meats. Long-term calorie-restricted diets, if not carefully designed, will likely lead to malnutrition and health hazards. And not eating carbohydrates for a long time will affect the mood and may lead to depression in severe cases. Even if the caloric restriction is beneficial to lifespan and health, it is difficult for most people to implement such restrictions in our lives for social, economic and medical reasons, especially, in a long term. To overcome the difficulties, more and more researches focused on developing medicines to mimic the beneficial effects of caloric restriction without actually restricting caloric intake. Such medicines are known as caloric restriction mimetics (CRM).

An effective caloric restriction mimetic (CRM) is a pharmaceutical or natural compound which would alter the key metabolic pathways involved in the effects of caloric restriction itself, therefore reproduce one or more principal biological effects of caloric restriction without reducing food intake, which may be especially suitable for mid-to late-life periods. Many putative calorie restriction mimetics have been found potentially useful in humans. For example, drugs that inhibit glycolysis (2-deoxyglucose), enhance insulin action (metformin), or affect stress signaling pathways (resveratrol), are being assessed as caloric restriction mimetics (Aging Cell. 2006 April; 5(2):97-108). Promising results have emerged from initial studies regarding physiological responses which resemble those observed in caloric restriction. Ultimately, lifespan analysis and expanded toxicity studies must be accomplished to fully assess the potential of any caloric restriction mimetics. Nonetheless, this strategy offers a very promising and expanding research endeavor.

Ergothioneine (ET) is a naturally occurring, water-soluble amino acid and is a thiourea derivative of histidine, containing a sulfur atom on the imidazole ring. It is mainly found in mushrooms, but also in king crab, and animals that have grazed on grasses containing Ergothioneine. Since humans or other vertebrates are unable to synthesize ergothioneine, it can only be acquired by diet. Ergothioneine has a wide tissue distribution in human, however, accumulates differentially in various tissues, with a more abundant accumulation in erythrocytes, liver, seminal fluid, bone marrow, eye lens, cornea, and kidney. Ergothioneine has demonstrated powerful antioxidant and cytoprotective properties in vitro. And it can protect mitochondrial function and gene stability, etc. Inventors found that Ergothioneine can activate PGC-1α through upregulating the Sirt1 expression, and further enhance mitochondrial biogenesis. Studies in animals and humans have found no toxicity or adverse effects to be associated with Ergothioneine administration, even at high doses, and recently, Ergothioneine (Tetrahedron, Paris, France) has attained European Food Safety Authority approval in the European Union and is generally recognized as a safe supplement by the Food and Drug Administration in the US (GRAS notice No. 734) (FEBS Letters 592 (2018) 3357-3366).

In the present invention, we have discovered a novel application of Ergothioneine as an effective caloric restriction mimetic that shows health benefits.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

This invention generally relates to compounds, compositions and methods for mimicking one or more biological benefits of caloric restriction in a mammal, comprising administering to the mammal in need thereof an effective amount of Ergothioneine, an analog or derivative thereof, or a pharmaceutically acceptable salt, acid, ester, polymer, analog or derivative thereof. According to the present invention, it was surprisingly found that Ergothioneine can function as a caloric restriction mimic, extending lifespan and providing other health benefits. It is believed that this invention is the first time to propose and conduct Ergothioneine as a caloric restriction mimic.

One aspect of the present invention provides a method for mimicking one or more biological benefits of caloric restriction in a mammal, comprising administering to the mammal an effective amount of Ergothioneine, an analog or derivative thereof, or a pharmaceutically acceptable salt, acid, ester, polymer, analog or derivative thereof.

In some embodiments, the Ergothioneine is D-Ergothioneine or L-Ergothioneine. In some further embodiments, the Ergothioneine is L-Ergothioneine.

In some embodiments, Ergothioneine is administered in an amount ranging from 0.1 mg/day-250 mg/day.

In some embodiments, Ergothioneine is administered in an amount ranging from 1 mg/day-200 mg/day, 2 mg/day-100 mg/day, 5 mg/day-50 mg/day or 5 mg/day-25 mg/day.

In some embodiments, the Ergothioneine is administered in a form of aqueous solution, aqueous suspension, capsule, drop, granule, liquid, powder, syrup, tablet, functionalized food, beverage, toothpaste, or sublingual articles.

In some embodiments, the mammal is human.

In some embodiments, the biological benefit includes, but not limited to, lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

In some embodiments, the Ergothioneine is administered orally, by intravenous injection, by intramuscular injection, intraperitoneally or sublingually.

In some embodiments, the Ergothioneine is prepared in a form of nutritional, drinking, or pharmaceutical composition, for use in a food, drink, nutritional, or pharmaceutical products.

In some embodiments, the Ergothioneine is administered as a dietary supplement or an ingredient in a food.

Another aspect of this invention relates to a composition capable of mimicking one or more biological benefits of caloric restriction, comprising an effective amount of Ergothioneine, an analog or derivative thereof, or a pharmaceutically acceptable salt, ester, polymer, acid, analog or derivative thereof.

In some embodiments, the Ergothioneine is L-Ergothioneine, L-Ergothioneine is administered in an amount ranging from 0.1 mg/day-250 mg/day.

In some embodiments, L-Ergothioneine is administered in an amount ranging from 1 mg/day-200 mg/day, 2 mg/day-100 mg/day, 5 mg/day-50 mg/day or 5 mg/day-25 mg/day.

In some embodiments, the biological benefit comprises the following: lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

In some embodiments, the Ergothioneine is administered in a form of aqueous solution, aqueous suspension, capsule, drop, granule, liquid, powder, syrup, tablet, functionalized food, beverage, toothpaste, or sublingual articles.

In some embodiments, the Ergothioneine is prepared in a form of nutritional, drinking, or pharmaceutical composition, for use in a food, drink, nutritional, or pharmaceutical products.

In some embodiments, the composition is a dietary composition or supplement.

A further aspect of the invention relates to use of Ergothioneine in manufacturing a composition capable of mimicking caloric restriction in a mammal.

In some embodiments, the Ergothioneine is L-Ergothioneine, L-Ergothioneine is administered in an amount ranging from 0.1 mg/day-250 mg/day.

In some embodiments, L-Ergothioneine is administered in an amount ranging from 1 mg/day-200 mg/day, 2 mg/day-100 mg/day, 5 mg/day-50 mg/day or 5 mg/day-25 mg/day.

In some embodiments, the biological benefit comprises the following: lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

In some embodiments, the Ergothioneine is administered in a form of aqueous solution, aqueous suspension, capsule, drop, granule, liquid, powder, syrup, tablet, functionalized food, beverage, toothpaste, or sublingual articles.

In some embodiments, the Ergothioneine is prepared in a form of nutritional, drinking, or pharmaceutical composition, for use in a food, drink, nutritional, or pharmaceutical products.

### BRIEF DESCRIPTION OF THE DRAWINGS

FIG. 1 is a graph of the abundance of LDL in A, B, C and D groups.

FIG. 2 is a graph of the abundance of HDL in A, B, C and D groups.

FIG. 3 is a graph of the abundance of HOMA-IR in A, B, C and D groups.

FIG. 4 is a graph of the abundance of pAKT/AKT in A, B, C and D groups.

FIG. 5 is a graph of the abundance of TNF-α in A, B, C and D groups.

FIG. 6 is a graph of the abundance of IL-6 in A, B, C and D groups.

FIG. 7 is a graph of the abundance of MDA in A, B, C and D groups.

FIG. 8 is a graph of the abundance of SOD in A, B, C and D groups.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for compositions and methods of mimicking one or more biological benefits of caloric restriction in a mammal, comprising administering to the mammal an effective amount of Ergothioneine, an analog or derivative thereof, or a pharmaceutically acceptable salt, ester, polymer, acid, analog or derivative thereof. Particularly, Ergothioneine can be used as a caloric restriction mimic, and it can be L-Ergothioneine or D-Ergothioneine. Moreover, Ergothioneine is administered in a variety of forms, such as aqueous solution, aqueous suspension, capsule, drop, granule, liquid, powder, syrup, tablet, functionalized food, beverage, toothpaste, or sublingual articles.

### Definitions

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “subject” or “patient” is used interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

As used herein, the term “treat”, “treating” or “treatment” and the like as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters, including the results of a physical examination, neurological examination, and/or psychiatric evaluations. Accordingly, the term “treating” or “treatment” includes the administration of L-Ergothioneine which may be in combination with other compounds or agents.

As used herein, the terms “preventing,” “prevention,” and the like are used generally to mean preventing or inhibiting deterioration or further deterioration of the visual system of an aging subject, as compared with a comparable visual system not receiving the synthetic retinal derivative.

As used herein, the term “effective amount” refers to an amount that is required to improve at least one symptom of a medical condition in an individual.

As used herein, the term “caloric restriction (CR)” and the like, refers to the dietary manipulations in yeast, worms, flies, rodents and other mammals, including humans, that result in increased life span and other biological benefits through reduction of caloric intake.

As used herein, the term “calorie restriction mimic” or “calorie restriction mimetic”, and the like, is pharmaceutical or natural compound or substance, mixture of compounds or substances that recapitulate calorie restriction conditions without the usual requirement for a reduction in calorie content of the diet.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “administration” refers to the process of delivering a disclosed combination, composition or kit to a subject. The combination, compositions or kits can be administered in a variety of ways, including orally, intragastrically, and parenterally (e.g., intravenous and intraarterial as well as other suitable parenteral routes), and the like.

As used herein, the term “pharmaceutically acceptable” means pharmaceutically, physiologically, alimentarily, or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

The following examples are illustrative of select embodiments of the present invention and are not meant to limit the scope of the invention.

### EXAMPLES

### Example 1

Mice group were named after the diet they consumed. Group A mice was fed ad libitum (control), and group B mice was on high-fat diet (HFD), Group C was fed restricted amount of the HFD diet to achieve caloric restriction (HFD+CR), and Group D was fed high-fat diet and supplemented with Ergothioneine (HFD+EGT), the concentration of Ergothioneine is 3.25 mg/kg. All groups of mice were housed individually, and fed for months of interventions and had free access to water. Health status was monitored during the intervention. At the end of each intervention, body composition, insulin sensitivity, blood glucose level, different blood parameters and lifespan of the mice were studied. After treatment, blood is taken to measure high-density lipoprotein (HDL), low-density lipoprotein (LDL) and HOmeostasis Model Assessment-Insulin Resistance (HOMA-IR). The HOMA-IR index was calculated using the following formula: HOMA-IR=[fasting glucose levels (mmol/L)]×[fasting serum insulin (mU/L)]/22.5. At the end of the experiment, AKT and phospho-AKT (Ser473) protein levels in skeletal muscle after 2 IU/kg insulin stimulation for 15 min were determined by immunoblotting (n=3-6). The concentrations of inflammatory cytokines including tumor necrosis factor-α (TNF-α) and interleukin (IL)-6, and oxidative stress markers including malonaldehyde (MDA) and superoxide dismutase (SOD) were detected by ELISA kits.

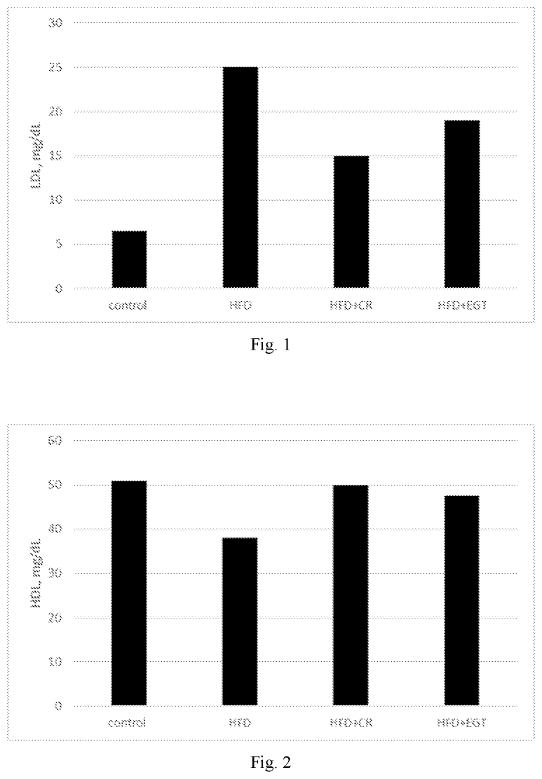

Data were collected and subject to statistical analysis. FIG. 1 is a graph of the abundance of LDL in A, B, C and D groups. The LDL level in B group (HFD) was significantly higher than that in A group (control), group C was fed restricted amount of the HFD diet to achieve caloric restriction (HFD+CR), and this method can decrease the level of LDL. But the way of caloric restriction will likely lead to malnutrition and cause health risks. Group D was fed high-fat diet and supplemented with Ergothioneine (HFD+EGT), and the level of LDL was significantly decreased which was close to the level of Group C. FIG. 2 is a graph of the abundance of HDL in A, B, C and D groups. The HDL level in B group (HFD) was significantly lower than that in A group (control), group C was fed restricted amount of the HFD diet to achieve caloric restriction (HFD+CR), and this method can increase the level of HDL. But the way of caloric restriction will likely lead to malnutrition and cause health risks. Group D was fed high-fat diet and supplemented with Ergothioneine (HFD+EGT), and the level of HDL was significantly increased which was close to the level of Group A.

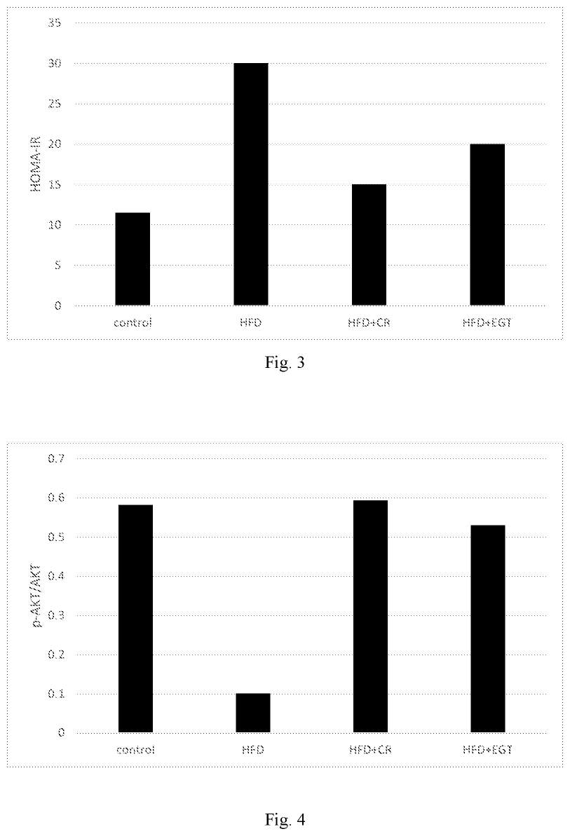

FIG. 3 is a graph of the abundance of HOMA-IR in A, B, C and D groups. As shown in FIG. 3, CR prevented the HFD-induced increase in fasting insulin. HFD+CR group and HFD+EGT group had lower HOMA-IR than HFD group which shows obvious effects of CR and EGT on fasting serum glucose or insulin. FIG. 4 is a graph of the abundance of phospho-AKT/AKT in A, B, C and D groups. AKT phosphorylation is obvious in Group C and D at the end of experiment. The insulin-stimulated AKT phosphorylation (p-AKT)/AKT relative protein expression in HFD+CR group and HFD+EGT group was higher than HFD group. It shows that supplementation with Ergothioneine can improve muscular insulin sensitivity.

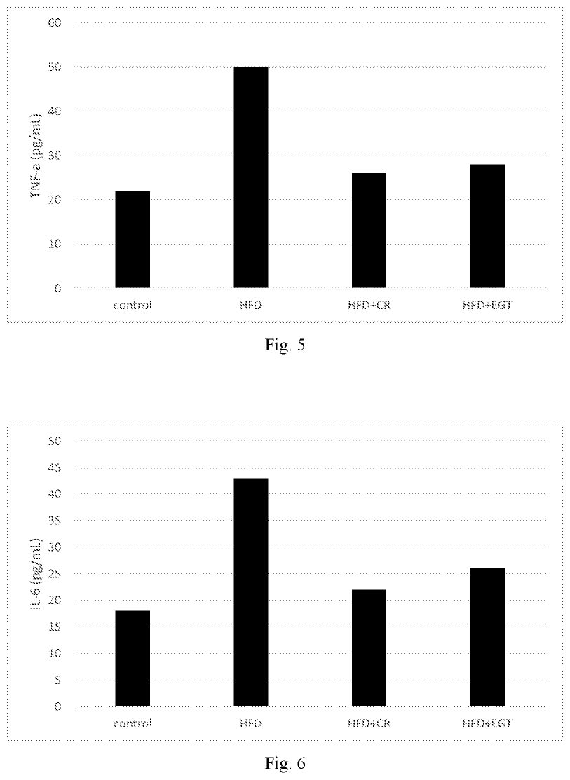

FIG. 5 is a graph of the abundance of TNF-α in A, B, C and D groups. FIG. 6 is a graph of the abundance of IL-6 in A, B, C and D groups. The contents of TNF-α (FIG. 5), IL-6 (FIG. 6) in the HFD group were higher than those of control group. Compared with HFD group, these levels in the HFD+EGT group were remarkably decreased. It shows that supplementation with Ergothioneine can decrease inflammation and/or mimic biological benefit of caloric restriction.

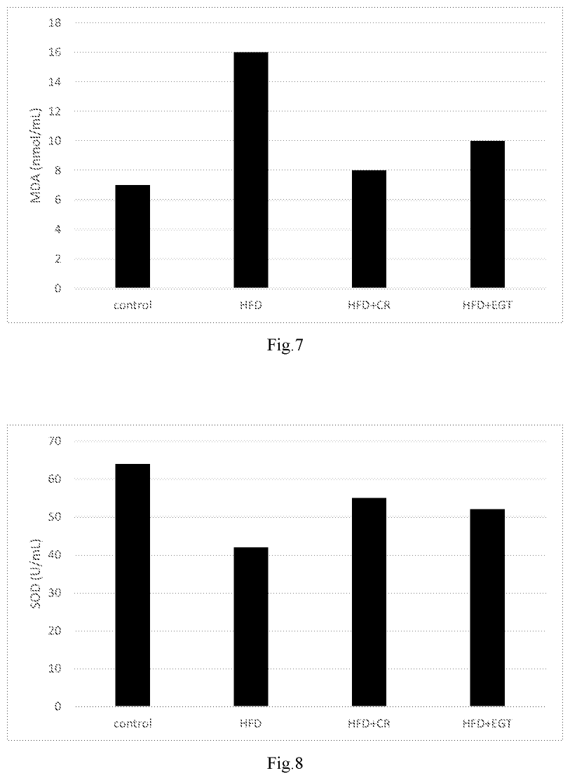

FIG. 7 is a graph of the abundance of MDA in A, B, C and D groups. FIG. 8 is a graph of the abundance of SOD in A, B, C and D groups. We found that EGT improves HFD-accelerated oxidative stress response, as demonstrated by the reduction of MDA expression (FIG. 7) and elevation of SOD expression (FIG. 8) compared with HFD group. It shows that supplementation with Ergothioneine can reduce oxidative stress and/or mimic biological benefit of caloric restriction.

In the present invention, the HOMA-IR is decreased by Ergothioneine to not higher than 28, 26, 24, 22, 20, 15. The p-AKT/AKT is increased by Ergothioneine to not lower than 0.25, 0.35, 0.40, 0.45, 0.53, 0.60. The LDL level is decreased by Ergothioneine to not higher than 24, 22, 20, 18, 15 mg/dL. The HDL level is increased by Ergothioneine to not lower than 40, 42, 44, 46, 48, 50 mg/dL. The level of TNF-α is decreased by Ergothioneine to not higher than 40, 38, 35, 32, 30, 28, 26 pg/mL. The level of IL-6 is decreased by Ergothioneine not higher than 38, 35, 30, 28, 26, 22 pg/mL. The level of MDA is decreased by Ergothioneine to not higher than 15, 13, 12, 11, 10, 8 nmol/mL. The level of SOD is increased by Ergothioneine to not lower than 45, 48, 50, 51, 52, 55 U/mL. Those data further indicate that ergothioneine may mimic one or more biological benefits of caloric restriction through mechanisms including lowering LDL, increasing HDL, improving insulin sensitivity, anti-inflammatory, anti-oxidative stress.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### OTHER EMBODIMENTS

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for mimicking one or more biological benefits of caloric restriction in a mammal, comprising administering to the mammal an effective amount of Ergothioneine, an analog or derivative thereof, or a pharmaceutically acceptable salt, acid, ester, polymer, analog or derivative thereof.

2. The method of claim 1, wherein the Ergothioneine comprises D-Ergothioneine or L-Ergothioneine.

3. The method of claim 1, wherein the Ergothioneine is L-Ergothioneine.

4. The method of claim 3, wherein L-Ergothioneine is administered in an amount ranging from 0.1 mg/day-250 mg/day.

5. The method of claim 3, wherein L-Ergothioneine is administered in an amount ranging from 2 mg/day-100 mg/day.

6. The method of claim 1, wherein the Ergothioneine is administered in a form of aqueous solution, aqueous suspension, capsule, drop, granule, liquid, powder, syrup, tablet, functionalized food, beverage, toothpaste, or sublingual articles.

7. The method of claim 1, wherein the biological benefit comprises the following: lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

8. The method of claim 1, wherein the Ergothioneine is administered orally, by intravenous injection, by intramuscular injection, intraperitoneally or sublingually.

9. The method of claim 1, wherein the Ergothioneine is prepared in a form of nutritional, drinking, or pharmaceutical composition, for use in a food, drink, nutritional, or pharmaceutical products.

10. The method of claim 1, wherein the Ergothioneine is administered as a dietary supplement or an ingredient in a food.

11. A composition capable of mimicking one or more biological benefits of caloric restriction, comprising an effective amount of Ergothioneine, an analog or derivative thereof, or a pharmaceutically acceptable salt, ester, polymer, acid, analog or derivative thereof.

12. The composition of claim 11, the Ergothioneine is L-Ergothioneine, L-Ergothioneine is administered in an amount ranging from 0.1 mg/day-250 mg/day.

13. The composition of claim 11, wherein the biological benefit comprises the following: lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

14. The composition of claim 11, wherein the Ergothioneine is administered in a form of aqueous solution, aqueous suspension, capsule, drop, granule, liquid, powder, syrup, tablet, functionalized food, beverage, toothpaste, or sublingual articles.

15. The composition of claim 11, wherein the Ergothioneine is prepared in a form of nutritional, drinking, or pharmaceutical composition, for use in a food, drink, nutritional, or pharmaceutical products.

16. Use of Ergothioneine in manufacturing a composition capable of mimicking one or more biological benefits of caloric restriction in a mammal.

17. The use of claim 16, wherein the Ergothioneine is L-Ergothioneine, L-Ergothioneine is administered in an amount ranging from 0.1 mg/day-250 mg/day.

18. The use of claim 16, wherein the biological benefit comprises the following: lowering heart rate, blood pressure, low-density lipoprotein, cholesterol and triglycerides; improving insulin sensitivity and normalize blood glucose; maintaining DNA integrity; reducing oxidative stress; decreasing inflammation; decreasing body temperature; reducing body fat mass, including visceral obesity, while increasing muscle mass; losing weight; enhancing the ability to engage in sports activities; improving brain function, including memory, cognition, and mood; stimulating growth factors; or improving lifespan and health.

19. The use of claim 16, wherein the Ergothioneine is administered in a form of aqueous solution, aqueous suspension, capsule, drop, granule, liquid, powder, syrup, tablet, functionalized food, beverage, toothpaste, or sublingual articles.

20. The use of claim 16, wherein the Ergothioneine is prepared in a form of nutritional, drinking, or pharmaceutical composition, for use in a food, drink, nutritional, or pharmaceutical products.
